(19) United States
(12) Patent Application Publication
(10) Pub. No.: US 2023/0056170 A1
(43) Pub. Date: Feb. 23, 2023

(54) Air purifier

(71) Applicants: Crawford Global Limited, Hong Kong (CN); ETI SOLID STATE LIGHTING (ZHUHAI) LTD., Zhuhai (CN)

(72) Inventors: Donglei WANG, Zhuhai (CN); Baiqiang HE, Zhuhai (CN); Li ZHANG, Zhuhai (CN); Songhong DING, Zhuhai (CN); Yingcai LV, Zhuhai (CN)

(21) Appl. No.: 17/892,160

(22) Filed: Aug. 22, 2022

(30) Foreign Application Priority Data
Aug. 23, 2021 (CN) 202110969871.4
Aug. 23, 2021 (CN) 202121997690.4

(51) Int. Cl. F24F 8/30; F24F 7/003; F24F 8/10

(52) U.S. Cl. CPC F24F 8/30; F24F 7/003; F24F 8/10

(57) ABSTRACT

The disclosure provides an air purifier. The air purifier includes a shell, a first plasma generator, a filter assembly, a second plasma generator, and a ventilation assembly. An air inlet and an air outlet are formed in the shell. A ventilation channel is formed between the air inlet and the air outlet of the shell. The first plasma generator, the filter assembly, the second plasma generator, and the ventilation assembly are separately arranged in the ventilation channel. In an air outlet direction of the ventilation channel, the first plasma generator is arranged between the air inlet and the filter assembly, and the second plasma generator is arranged between the filter assembly and the air outlet. By adopting the above structure, the first plasma generator is arranged between the air inlet and the filter assembly, and a plasma wall is formed on the periphery of the filter assembly.

### CROSS-REFERENCE TO RELATED PRESENT INVENTION(S)

The disclosure claims priority to and the benefit of Chinese Patent Present invention No.202110969871.4 and No.202121997690.4, filed in the China National Intellectual Property Administration (CNIPA) on 23 Aug. 2021, which are incorporated herein by reference in its entirety.

### TECHNICAL FIELD

The disclosure relates to the technical field of environment protection, and in particular, to an air purifier.

### BACKGROUND

At present, indoor environments of human habitation often suffer from various types of pollution, some of which cause the symptoms of dizziness, nausea, and the like of people, affecting the quality of life and the health of people.

In order to improve the quality of life, ionic air purifiers are gradually widely used to purify indoor air. At present, the ionic air purifiers on the market include negative-ion generators and plasma purifiers. The negative-ion generator adsorb charged particles in the air by generating ions, so that the particles attracted to each other become heavier and fall from the air, so as to realize air purification. The plasma purifiers generate high-energy electrons under the action of an external electric field. When the high-energy electrons collide with pollutant molecules in the air, a series of physical and chemical reactions will be induced, so that those macromolecular pollutants can be transformed into small-molecule safe substances, thereby achieving the purposes of purifying the air and removing odor.

However, the existing plasma air purifiers usually have a group of plasma generators at an air inlet or an air outlet, which makes the plasma distribution in the air uneven, or the amount of plasma in the air is not enough, so the effects of disinfecting and sterilizing cannot be achieved. Or, the ozone generated during working of the plasma air purifiers exceeds the standard, which causes secondary pollution of the air and makes people feel uncomfortable.

In addition, a fan assembly is arranged in an ionic air purifier. The indoor air is pumped into the purifier through a ventilation assembly of the fan assembly, is circulated along an air channel in the purifier, and then is exhausted into the indoor air after being disinfected and sterilized in the air channel of the purifier. Based on the layout of the components in the air purifier, other components are arranged in the air channel of the existing purifier, so that the air flow in the air channel will encounter corners to produce vortices to form resistance and air flow noise. Therefore, the air volume of the air outlet is reduced, the air outlet is uneven, and the noise reduces the use experience and reduces the good impression of a user.

### SUMMARY

A main objective of the disclosure is to provide an air purifier including at least groups of plasma generators.

In order to achieve the abovementioned main objective, in an embodiment mode, the air purifier provided by the disclosure includes a shell, a first plasma generator, a filter assembly, a second plasma generator, and a ventilation assembly. An air inlet and an air outlet are formed in the shell. A ventilation channel is formed between the air inlet and the air outlet of the shell. The first plasma generator, the filter assembly, the second plasma generator, and the ventilation assembly are separately arranged in the ventilation channel. In an air outlet direction of the ventilation channel, the first plasma generator is arranged between the air inlet and the filter assembly, and the second plasma generator is arranged between the filter assembly and the air outlet.

It can be seen that at least two groups of plasma generators are arranged in the air purifier. The first plasma generator is arranged close to the air inlet, and the plasma generator close to the air inlet is arranged between the air inlet and the filter assembly, so that a plasma wall is formed on the periphery of the filter assembly. In the air outlet direction of the ventilation channel, under the action of the ventilation assembly, air passes through the plasma wall and the filter assembly from the air inlet in sequence. The air entering the purifier is disinfected and sterilized, and the second plasma generator is arranged close to the air outlet. The plasma released by the second plasma generator forms a plasma jet with the disinfected and sterilized air, and is sent into a room. With the work of the air purifier, positive ions and negative ions are continuously released into the air, and the positive ions and the negative ions in the indoor air outside the purifier produce and release huge energy at the moment of neutralizing positive and negative charges in the air, resulting in the change of a bacterial structure around or the conversion of energy, resulting in the death of bacteria. The plasma jet in the indoor air outside the purifier realizes a sterilization effect thereof. In the air circulation realized by the purifier, the two plasma generators respectively sterilize the air inside and outside the purifier, so that the air is purified more thoroughly.

In an embodiment mode, the first plasma generator includes a plurality of pairs of first plasma emitters, and the second plasma generator includes a plurality of pairs of second plasma emitters. In a first projection direction, the plurality of pairs of first plasma emitters and the plurality of pairs of second plasma emitters are arranged in the circumferential direction of the ventilation channel in a staggered mode.

It can be seen that in the circumferential direction of the ventilation channel, the plurality of pairs of emitters of the two plasma generators are arranged in a staggered mode, so that the plasma jet generated by the second plasma generator further sterilizes the air flowing from the air inlet chamber to the air outlet chamber, meanwhile, the plasmas finally released into a room are distributed more evenly, and the disinfection and sterilization effects are better.

In an embodiment mode, in the vertical direction, the air outlet is provided above the air inlet. The shell includes an air inlet chamber and an air outlet chamber. The air inlet chamber is arranged above the air inlet chamber. The air inlet chamber is communicated with the air inlet. The air outlet chamber is communicated with the air outlet. The ventilation assembly is arranged to send air of the air inlet chamber into the air outlet chamber. The first plasma generator and the filter assembly are separately arranged in air inlet chamber. The second plasma generator is arranged in the air outlet chamber.

It can be seen that the air outlet is located above the air inlet, so that the ventilation assembly extends in the vertical direction, the air released from the air outlet does not need to turn and then is diffused upward, and indoor plasmas are distributed more evenly. The ventilation assembly is located between the air inlet chamber and the air outlet chamber, which ensures that the air is pumped into the purifier, shortens the distance between the ventilation assembly and the air outlet, and accelerates the exhaust speed of the air in the purifier.

In an embodiment mode, the shell includes side plate and a base. The side plate is connected to the base along the circumferential direction of the base. A plurality of through holes are formed in the side plate. The plurality of through holes form the air inlet. A horizontal air inlet section is formed between the side plate and the filter assembly in the air inlet chamber. In the horizontal air inlet section, the first plasma generator is arranged between the side plate and the filter assembly in the air outlet direction of the horizontal air inlet section.

It can be seen that the side plate are arranged in the circumferential direction of the base. A plurality of through holes are formed in the side plate, so that an air inlet through which air can enter in all directions at 360° is formed in the shell, and the air inlet volume of the purifier is expanded. The first plasma generator is arranged between the side plate and the filter assembly, and a plasma wall is formed between the air outlet and the filter assembly to disinfect and sterilize the air at the air inlet first, and then a purification effect of the air is ensured through the filter assembly. Negatively charged particles generated by the plasma generator can also supplement charges to a filter screen in the filter assembly, so that the filter assembly maintains an efficient filtering effect. It is to be noted that positive and negative ions can be generated at the same time when the plasma generators work, and the generated positive and negative ions can be used to disinfect and sterilize virus and bacteria in the filter assembly. In the solution, the number of the generated negative ions will be greater than the number of the generated positive ions during actual work of the selected plasma generators, so the excessive negative ions can also supplement charges to the filter screen in the filter assembly. Therefore, the filter assembly maintains an efficient filtering effect.

In an embodiment mode, in the vertical direction, the first plasma generator is arranged above the filter assembly. Based on the base, the maximum arrangement height of the plurality of through holes in the side plate is greater than the arrangement height of the first plasma generator.

It can be seen that, the first plasma generator is arranged above the filter assembly, and the height of the air inlet is greater than the arrangement height of the first plasma generator, so that the ventilation channel is narrowed gradually, and the sterilization effect is better.

In an embodiment mode, the filter assembly is in the shape of a circular cylinder, and a vertical ventilation section is formed in an inner circle of the filter assembly. In the air outlet direction of the ventilation channel, the vertical ventilation section is located downstream of the horizontal air inlet section. The filter assembly includes a first filter layer, a second filter layer, and a third filter layer. In the radial direction of the filter assembly, the second filter layer is wrapped outside the third filter layer, and the first filter layer is wrapped outside the second filter layer. The first filter layer is a nylon filter screen, the second filter layer is a High-Efficiency Particulate Air (HEPA) filter, and the third filter layer is an activated carbon filter layer.

It can be seen that the air after passing through the plasma wall is filtered by using a composite filter assembly. The nylon filter screen of the first filter layer can effectively filter large particles, such as pollen, hair, animal dander, etc. The HEPA filter of the second filter layer can filter particles with smaller particle sizes. The third filter layer is activated carbon, which can remove odor, Volatile Organic Compounds (VOC), etc., so as to improve the air purification effect. In addition, the first plasma generator polarizes the particles and pollutants entering the air so as to be absorbed into the filter assembly more easily in addition to sterilizing the incoming air, which improves the interception efficiency of the filter assembly, polarizes the HEPA filter layer, improves the adsorption efficiency of the filter assembly, and prolongs the effective life.

In an embodiment mode, the ventilation assembly covers a ventilation opening, facing the ventilation assembly, of the air inlet chamber.

It can be seen that the wind wheel covers the ventilation opening, facing the air outlet chamber, of the air inlet chamber, so as to pump the air in the air inlet chamber to the air outlet chamber as far as possible.

In an embodiment mode, a flow guide assembly is arranged in the air inlet chamber. The flow guide assembly includes a flow guide frame. The ventilation channel is formed on the periphery of the flow guide frame. The flow guide frame is in a circular truncated cone shape. In the air outlet direction of the ventilation channel, the radial width of a lower bottom surface of the flow guide frame is greater than the radial width of an upper top surface of the flow guide frame. The lower bottom surface of the flow guide frame faces the ventilation assembly. The upper top surface of the flow guide frame faces the air outlet.

It can be seen that the ventilation assembly pumps the air in the air inlet chamber to the air outlet chamber. The upper top surface with a smaller radial width of the flow guide frame faces the air outlet, while the lower bottom surface with a larger radial width of the flow guide frame faces the ventilation assembly, so that in the air outlet direction of the ventilation channel, the air pumped by the ventilation assembly is gradually gathered at the periphery of the flow guide frame along a side conical surface of the flow guide frame and flows to the air outlet, and the air dispersed by the ventilation assembly is gathered under the flow guiding effect of the flow guide frame, which increases the air outlet volume, meanwhile, a vortex is prevented from forming at the center of the air flow since the flow guide frame is arranged at the center of the air flow, the loss of the air volume is reduced, and meanwhile, the noise generated due to a vortex phenomenon is reduced.

The shell, the ventilation assembly, and the flow guide assembly form an air channel assembly. It can be seen that the ventilation assembly is arranged between the air outlet and the air inlet, so that the ventilation assembly is arranged in the middle of the ventilation channel, while the flow guide frame is arranged between the ventilation assembly and the air outlet. Under the action of the ventilation assembly, when the air flows at a high speed in the ventilation channel and passes through the flow guide frame, since the upper top surface with a smaller radial width of the flow guide frame faces the air outlet, while the lower bottom surface with a larger radial width of the flow guide frame faces the ventilation assembly, in the air outlet direction of the ventilation channel, a circulation space of the air outlet chamber increases gradually, the air pumped by the ventilation assembly is gradually gathered at the periphery of the flow guide frame along a side conical surface of the flow guide frame and flows to the air outlet, and the air diffused by the ventilation assembly is gathered under the flow guiding effect of the flow guide frame, which increases the air outlet volume, meanwhile, the flow guide frame is arranged at the center of the air flow since the ventilation channel is formed on the periphery of the flow guide frame, a vortex is prevented from forming at the center of the air flow, the loss of the air volume is reduced, and meanwhile, the noise generated due to a vortex phenomenon is reduced.

In an embodiment mode, the flow guide assembly includes a first mounting frame. The first mounting frame is arranged between the flow guide frame and the ventilation assembly. A flow guide section is formed at one end, facing the ventilation assembly, of the first mounting frame. The flow guide section is shaped like a circular truncated cone. In the air outlet direction of the ventilation channel, the radial width of the flow guide section increases gradually.

It can be seen that the flow guide section is arranged between the flow guide frame and the ventilation assembly. The radial width of the flow guide section gradually increases with the air outlet direction of the air outlet chamber, so that the air pumped by the ventilation assembly flows along the outer contour of the flow guide section. Meanwhile, after the air turns at the connection between the flow guide section and the flow guide frame, more air accumulates on the side conical surface of the flow guide frame for climbing, so as to reduce the resistance and noise formed when the air turns, and the seamless adjacency between the flow guide frame and the first mounting frame makes the connection more smooth.

In an embodiment mode, the lower bottom surface of the flow guide frame is adjacent to the side wall, facing the flow guide frame, of first mounting frame. The radial width of the lower bottom surface of the flow guide frame is equal to the maximum radial width of the flow guide section.

It can be seen that the first mounting frame is adjacent to the flow guide frame to form a completed flow guide shuttle structure. In the air outlet chamber, the air flow flows in the air outlet direction of the ventilation channel on the periphery of the flow guide shuttle structure. The flow guide shuttle structure is located at the center of the air flow of the air outlet chamber to prevent a vortex from forming at the center of the air flow during the flowing of the air flow, so as to reduce the loss of air volume, effectively improve the air speed, and meanwhile, reduce the generation of noise.

In an embodiment mode, the flow guide assembly includes a deflector. The deflector is annular. A first end, along the air outlet direction of the ventilation channel, of the deflector is arranged close to the air inlet chamber. A second end, along the air outlet direction of the ventilation channel, of the deflector is arranged away from the air inlet chamber. The first end of the deflector is arranged along the circumferential direction of the air inlet chamber close to the ventilation opening of the air outlet chamber. The second end of the deflector is arranged along the circumferential direction of the inner side wall of the shell. In the air outlet direction of the ventilation channel, the radial width of the deflector increases gradually from the first end of the deflector to the second end of the deflector.

It can be seen that the air in the air outlet chamber is fully diffused into the air inlet chamber under the action of the deflector.

The ventilation assembly is arranged in the deflector. Further, the ventilation assembly is arranged in the inner circle of the deflector.

In an embodiment mode, the first plasma generator is arranged on the side wall, deviating from the ventilation assembly, of the deflector.

It can be seen that the deflector achieves, in the air inlet chamber, an effect of guiding on the flowing of the air in the air outlet chamber, and also acts as a mounting carrier of the first plasma generator, so that the layout of the components in the purifier is more reasonable.

In an embodiment mode, the flow guide assembly further includes a second mounting frame. The second mounting frame is annular. The first mounting frame is arranged in the inner circle of the second mounting frame, and the second mounting frame is adjacent to the inner side wall of shell. The ventilation channel is located between the first mounting frame and the second mounting frame.

In an embodiment mode, at least two bumps are formed on the outer side wall of the flow guide frame. At least two connecting blocks are connected between the first mounting frame and second mounting frame. A mounting groove is formed in each of connecting blocks. One of the bumps is correspondingly arranged in one of the mounting grooves.

It can be seen that the second mounting frame is connected to an inner side wall of the shell. The flow guide frame is connected to the second mounting frame through a structure that the bumps are in fit connection with the mounting grooves, so as to realize the mounting of the flow guide frame in the shell.

In an embodiment mode, the ventilation assembly includes a driving device and a wind wheel. The driving device drives the wind wheel to rotate. The driving device is arranged in the inner circle of the flow guide section.

It can be seen that the driving device is located in the flow guide section, so that the layout of the components in the air outlet chamber is more reasonable.

In an embodiment mode, the wind wheel includes a plurality of blades. An air inlet channel is formed between two adjacent blades. The ventilation channel penetrates through the air inlet channel, and the side wall, facing the flow guide section, of the blade is in an arc.

It can be seen that an arc-shaped side wall on the blade is more closely fitted with the outer contour of the flow guide section, so that the air flows better along the outer contour of the flow guide section, is dispersed to the periphery of the first mounting frame, and then climbs along the flow guide section and the outer side wall of the flow guide frame. In an embodiment mode, a plurality of pairs of second plasma emitters are arranged on the flow guide frame.

It can be seen that the air of the air outlet chamber is gathered on the periphery of the flow guide frame, and the second plasma emitters are arranged on the flow guide frame, so that the plasmas generated by the second plasma emitters can be dispersed in air and flows out of the purifier with air, thereby disinfecting and sterilizing indoor air.

In an embodiment mode, the flow guide frame includes an upper flow guide frame and a lower flow guide frame. The upper flow guide frame and the lower flow guide frame are respectively shaped like circular truncated cones. The lower bottom surface of the upper flow guide frame is relatively butted with the upper top surface of the lower flow guide frame. The radial width of the lower bottom surface of the upper flow guide frame is equal to the radial width of the upper top surface of the lower flow guide frame. A first mounting chamber is formed in the upper flow guide frame. A high-voltage power supply assembly of the second plasma generator is arranged in the first mounting chamber. A second mounting chamber is formed in the lower flow guide frame. A high-voltage power supply assembly of the first plasma generator and a control assembly are arranged in the second mounting chamber. The control assembly is connected to the first plasma generator and the second plasma generator.

It can be seen that electrical elements or the control assembly are respectively mounted in the first mounting chamber and the second mounting chamber, which can reduce the influence of the mounting of the electrical elements on the ventilation volume of the ventilation channel, and meanwhile, realizes insulation protection on the electrical elements.

In an embodiment mode, the air outlet chamber is in a circular truncated cone shape. In the air outlet direction of ventilation channel, the radial width of the air outlet chamber decreases gradually; and/or the air outlet is located at the top of the shell, and in the vertical direction, the air outlet is provided above the air inlet.

It can be seen that the flow guide frame is arranged in the air outlet chamber. The width of the flow guide frame decreases gradually in the air outlet direction of the ventilation channel, while the radial width of the air outlet chamber decreases gradually in the same direction. Under the guiding action of the side wall of the air outlet chamber, the air climbs along the outer side wall of the flow guide frame, so that the air is gathered and the air outlet volume is increased. The air flows in the air outlet direction of the ventilation channel, and is sprayed out from the air outlet at the top, so that the air at the air outlet does not need to be diffused after turning. Therefore, the air outlet volume reaches the maximum.

In an embodiment mode, an air outlet grille is arranged at the air outlet. A plurality of air outlets are formed in the air outlet grille. The side walls of the air outlets are planes. The side walls of the air outlets extend in the vertical direction.

It can be seen that the air is guided by an inclined plane of a side conical surface of the flow guide frame first in the air outlet chamber, and then passes through a straight plane in the air outlet grille, so as to reduce the cases that air encounters a turn during high-speed flow, thereby avoiding the resistance and noise generated due to turning.

In an embodiment mode, the shell is in a circular truncated cone shape; and the width of the bottom of the shell is greater than the width of the top of the shell.

It can be seen that the circular truncated cone-shaped shell makes the shell have a better anti-dumping effect.

### BRIEF DESCRIPTION OF THE DRAWINGS

FIG. 1 shows a three-dimensional diagram of an embodiment of an air purifier of the disclosure.

FIG. 2 shows a sectional view of the embodiment of the air purifier of the disclosure.

FIG. 3 shows an exploded view of components in an upper shell in the embodiment of the air purifier of the disclosure.

FIG. 4 shows an exploded view of components in a lower shell in the embodiment of the air purifier of the disclosure.

FIG. 5 shows a schematic diagram of the connection of a flow guide frame and a mounting frame in the embodiment of the air purifier of the disclosure.

FIG. 6 shows a vertical view of the interior of the embodiment of the air purifier of the disclosure.

The disclosure will be further described below with reference to the accompanying drawings and specific embodiments.

### DETAILED DESCRIPTION OF THE EMBODIMENTS

The air purifier of the disclosure is applied to a disinfection and sterilization process for indoor air. Two plasma generators are arranged in the air purifier of the disclosure. In an air outlet direction of a ventilation channel in the air purifier, a first plasma generator disinfects and sterilizes the air at an air inlet, and then a second group of plasma generator uniformly distributes ions in filtered air after impurities are filtered through a filter assembly, finally, plasma-containing air is released into a room through the air outlet, and the indoor air is disinfected and sterilized. Two plasma generators are arranged at different positions, so that different disinfection and sterilization effects are achieved, and the disinfection and sterilization effects are better.

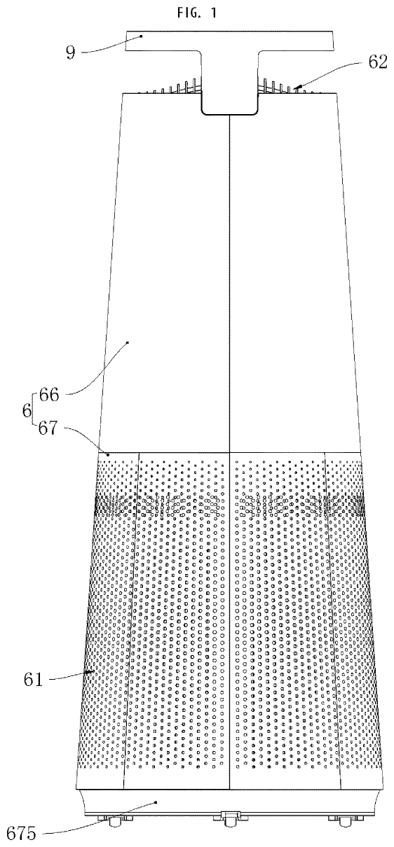

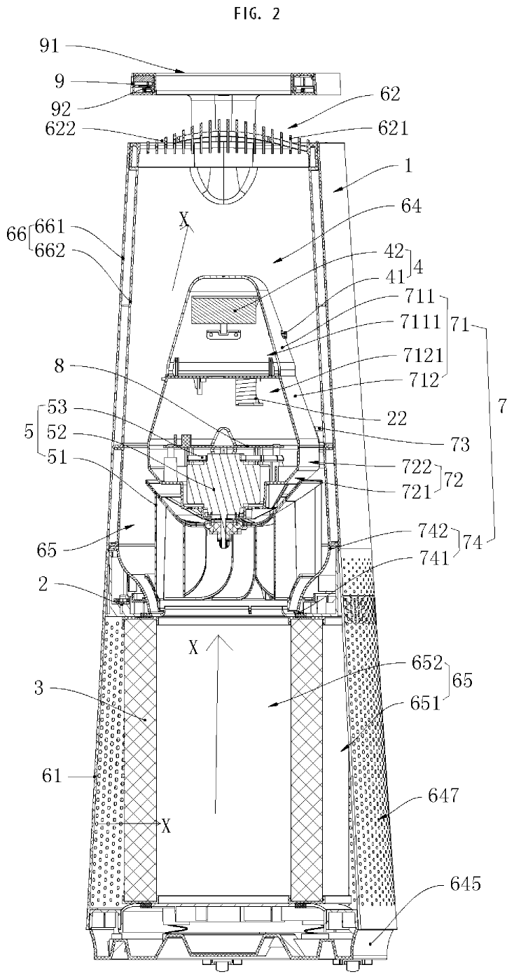

Referring to FIG. 1 and FIG. 2, the air purifier includes an air channel assembly 1, a first plasma generator 2, a filter assembly 3, and a second plasma generator 4. The air channel assembly 1 includes a shell 6, a ventilation assembly 5, and a flow guide assembly 7. The shell 6 includes an air inlet chamber 63, an air outlet chamber 64, an air inlet 61, an air outlet 62, and a ventilation channel 65. The air inlet chamber 63 is communicated with the air inlet 61. The air inlet chamber 63 is communicated with the air outlet chamber 64. The air outlet chamber 64 is communicated with the air inlet 62. The ventilation channel 65 is formed between the air inlet 61 and the air outlet 62. The ventilation assembly 5 is arranged to send the air of the air inlet chamber 63 to the air outlet chamber 64. The flow guide assembly 7 is arranged in the air outlet chamber 64. The flow guide assembly 7 is arranged between the air outlet 62 and the ventilation assembly 5. The ventilation channel 65 is formed on the periphery of the flow guide assembly 7. The first plasma generator 2, the filter assembly 3, and the second plasma generator 4 are separately are arranged in the ventilation channel 65. In an air outlet direction of the ventilation channel 65, the first plasma generator 2, the filter assembly 3, and the second plasma generator 4 are arranged in the shell 6 in sequence. The first plasma generator 2 and the filter assembly 3 are separately arranged in the air inlet chamber 63, and the second plasma generator is arranged in the air outlet chamber 64. The ventilation channel 65 may extend in the horizontal direction or extend in the vertical direction. However, no matter in which direction the ventilation channel 65 extends, in the ventilation channel 65, air passes through the air inlet 61, the first plasma generator 2, the filter assembly 3, the second plasma generator 4, and the air outlet 62 in sequence, so that negative and positive ions released by the first plasma generator 2 disinfect and sterilize the air at the air inlet 61 first, and then the air flows to the air outlet 62 under a guiding action of the flow guide assembly 7 after large and small granular objects, odor, and the like in the air are filtered by the filter assembly 3. Since the second plasma generator 4 is arranged between the air outlet 62 and the ventilation assembly 5, when the air flows to the air outlet chamber 64, the negative and positive ions released by the second plasma generator 4 are dispersed in the air outlet chamber 64 and are dispersed outside the purifier with air through the air outlet 62, so as to disinfect and sterilize indoor air outside the purifier.

In the present embodiment, the air outlet 62 is located at the top of the shell. In the vertical direction, the air outlet 62 is provided above the air inlet 61, and the air inlet 61 is formed in an outer side surface of the shell 6.

In the present embodiment, the shell 6 includes an upper shell 66 and a lower shell 67. The upper shell 66 is connected to the lower shell 67. In the vertical direction, the upper shell 66 is arranged above the lower shell 67. The air inlet chamber 63 is formed in the lower shell 67. The air outlet chamber 64 is arranged in the upper shell 66, so that the upper shell 66 is also provided above the air inlet chamber 67.

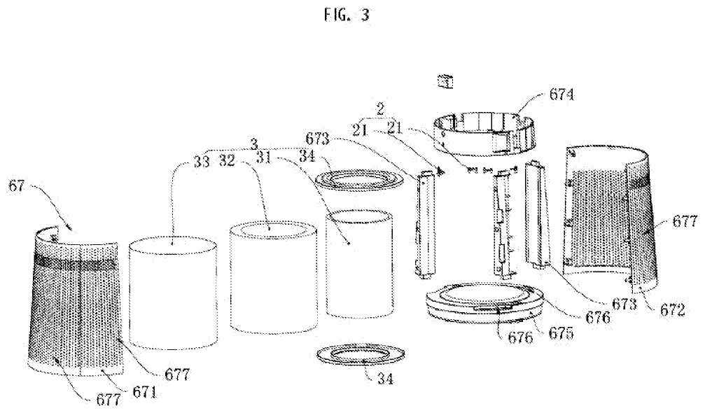

Referring to FIG. 3, the lower shell 67 includes a first side plate 671, a second side plate 672, a plurality of supporting posts 673, a fixed ring 674, and a base 675. A plurality of fixing grooves 676 are formed in the base 675. The plurality of supporting posts 673 are respectively arranged in the plurality of fixing grooves 676 in one-to-one correspondence. A first end, in the length direction of the supporting posts 673, of one supporting post 673 corresponds to one fixing groove 676. The supporting post 673 is arranged between the base 675 and the fixed ring 674. A second end, in the length direction of the supporting posts 673, of one supporting post 673 is connected to the fixed ring 674. The first side plate 671 and the second side plate 672 are connected to form a ring through a buckle structure. The first side plate 671 and the second side plate 672 are respectively connected to the base 675 in the circumferential direction of the base 675. The first side plate 671 and the second side plate 672 are respectively connected to the plurality of supporting posts 673, so as to realize the connection of the first side plate 671 and the second side plate 672 on the base 675. In the vertical direction, the fixed ring 674 is arranged above the filter assembly 3.

A plurality of through holes 677 are respectively formed in the first side plate 671 and the second side plate 672. The first side plate 671 and the second side plate 672 are connected to form a ring, and the air inlet chamber 63 is formed in an inner circle of the ring formed after the first side plate 671 and the second side plate 672 are connected. The through holes 677 in the first side plate 671 and the second side plate 672 are combined to form a huge air inlet 61. Air can enter through the air inlet 61 in all directions at 360° of the purifier, so as to increase the air inlet volume.

The first plasma generator 2 includes a plurality of pairs of first plasma emitters 21. The plurality of pairs of first plasma emitters 21 are arranged along the periphery of the filter assembly 3 at equal distance. Each pair of first plasma emitters 21 includes a positive ion emitter and a negative ion emitter. The plurality of pairs of first plasma emitters 21 are arranged on the fixed ring 674.

The filter assembly 3 is arranged in the air inlet chamber 63 in the lower shell 67. The plurality of pairs of first plasma emitters 21 in the first plasma generator 2 are separately arranged between the first side plate 671 and the filter assembly 3, and between the second side plate 672 and the filter assembly 3.

In the present embodiment, the filter assembly 3 is in the shape of a circular cylinder. The filter assembly 3 includes a first filter layer 31, a second filter layer 32, and a third filter layer 33. In the radial direction of the filter assembly 3, the second filter layer 32 is wrapped outside the third filter layer 33, and the first filter layer 31 is wrapped outside the second filter layer 32. The three filter layers are connected to form a composite filter layer through upper and lower mounting rings 34, and the mounting ring 34 located at the bottom of the purifier is arranged on the base 375. The first filter layer 31 is a nylon filter screen, the second filter layer 32 is a HEPA filter, and the third filter layer 33 is an activated carbon filter layer. The filter assembly 3 includes a plurality of layers of filter materials, so that an air purification effect is better.

The ventilation channel 65 is provided with a horizontal air inlet section 651 and a vertical air inlet section 652 in the air inlet chamber 63. The air inlet direction of the horizontal air inlet section 651 is perpendicular to the air inlet direction of the vertical air inlet section 652. The horizontal air inlet section 651 extends in the horizontal direction, and the vertical air inlet section 652 extends in the vertical direction. In the air outlet direction in the air inlet chamber 63, the vertical air inlet section 652 is arranged downstream the horizontal air inlet section 651. The path of the ventilation channel 65 in the horizontal air inlet section 651 in the air inlet chamber 63 is an inner circle from the air inlet 61 to the filter assembly 3, and the vertical air inlet section 652 is located in the inner circle of the filter assembly 3. The vertical air inlet section 652 is communicated with the air outlet chamber 64 in the vertical direction. In the air outlet direction X of the ventilation channel 65, the air outlet chamber is located downstream the vertical air inlet section 652. In the air outlet direction of the horizontal air inlet section 651, the plurality of pairs of first plasma emitters in the first plasma generator 2 are separately arranged between the first side plate 671 and the filter assembly 3 or between the second side plate 672 and the filter assembly 3. In the air outlet direction X of the ventilation channel 65, air enters the horizontal air inlet section 651 from the air inlet 61, then passes through the vertical air inlet section 652, and then enters the air outlet chamber 64 of the ventilation channel 65 through the ventilation assembly 5. In the horizontal air inlet section 651, the first plasma generator 2 is arranged between the air inlet 61 and the filter assembly 3, so that a plasma wall is formed between the air inlet 61 and the filter assembly 3. The air at the air inlet 61 is filtered through the filter assembly 3 after being disinfected and the sterilized, and then enters the inner circle of the filter assembly 3, that is, enters the vertical air inlet section 652. The plurality of pairs of first plasma emitters 21 are arranged between the air inlet 61 and the filter assembly 3, and negative ions emitted from the first plasma emitters 21 supplement the charges of the filter assembly 3, so that the filter effect is better.

In the present embodiment, based on the base 675, the arrangement height of the plurality of through holes 677 in the first side plate 671 and the arrangement height of the plurality of through holes 677 in the second side plate 672 are respectively greater than the arrangement height of the plurality of pairs of first plasma emitters 21 of the first plasma generator 2, so that the ventilation channel 65 is gradually narrowed in the horizontal air inlet section 651. Therefore, the sterilization effect is better.

The upper shell 66 includes an upper shell 661 and a middle shell 662. The upper shell 661 is connected to each of the first side plate 671 and the second side plate 672 in the circumferential direction of the lower shell 67, so that the upper shell 661 encloses to form a ring. The middle shell 662 is arranged between the upper shell 661 and the ventilation channel 65. The middle shell 662 fits the inner side wall of the upper shell 661, so that the middle shell 662 also encloses to form a ring, and the air inlet chamber 63 is formed in the ring enclosed by the middle shell 662.

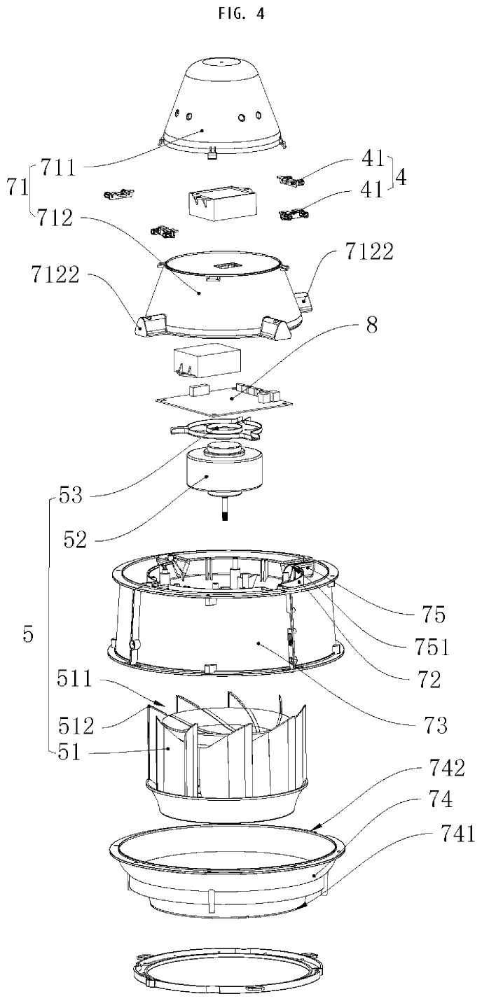

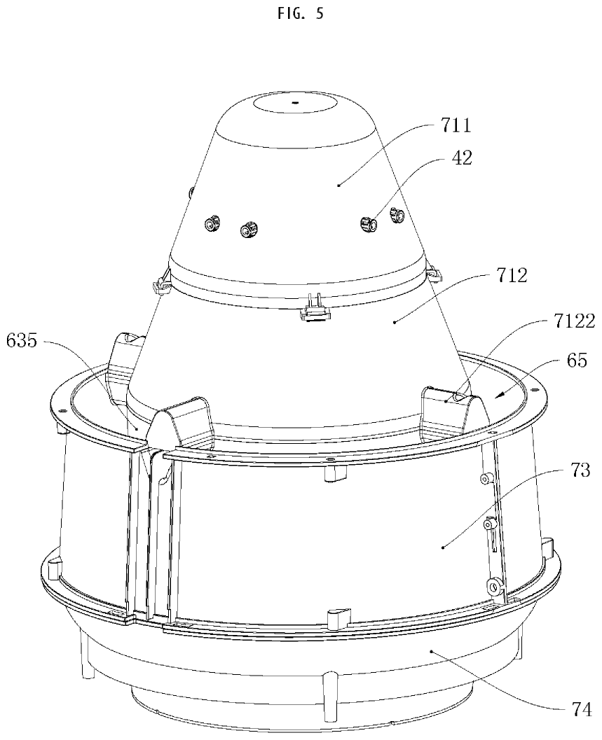

Referring to FIG. 4 and FIG. 5, the flow guide assembly 7 is arranged in the air inlet chamber 63. The flow guide assembly 7 includes a flow guide frame 71, a first mounting frame 72, a second mounting frame 73, and a deflector 74. The flow guide frame 71 is fixed to the interior of the air inlet chamber 63 through the first mounting frame 72 and the second mounting frame 73. The deflector 74 is arranged between the first mounting frame 72 and the air inlet chamber 63. The deflector 74 is arranged to guide the flowing of the air in the air inlet chamber 63.

In the present embodiment, the deflector 74 is annular. The air inlet chamber 63 faces a ventilation opening 631 of the air outlet chamber 64. A first end 741, along the air outlet direction of the ventilation channel 65, of the deflector 74 is arranged close to the air inlet chamber 63. A second end 742, along the air outlet direction of the ventilation channel 65, of the deflector 74 is arranged away from the air inlet chamber 63. The first end 741 of the deflector 74 is arranged along the circumferential direction of the air inlet chamber 63 close to the ventilation opening 631 of the air outlet chamber 64. The second end 742 of the deflector 74 is connected to the middle shell 662 in the circumferential direction of the middle shell 662. In the air outlet direction X of the ventilation channel 65, the radial width of the deflector 74 increases gradually from the first end 741 of the deflector 74 to the second end 742 of the deflector 74.

In the present embodiment, the ventilation assembly 5 is arranged in the inner circle of the deflector 74. The plurality of pairs of first plasma emitters 21 in the first plasma generator 2 are arranged on the side wall, deviating from the ventilation assembly 5, of the deflector 74. The radial width of the ventilation opening, close to the air outlet chamber 64, of the vertical air inlet section 652 is less than the width of the air outlet chamber 64. The ventilation assembly 5 located in the deflector 74 covers the ventilation opening 631. Under the guiding action of the deflector 74, the radial width of the deflector 74 increases gradually in the air outlet direction X of the ventilation channel 65. The ventilation assembly 5 can pump the air in the vertical air inlet section 652 to the air outlet chamber 64 and evenly distributes the air in the ventilation channel 65. The plurality of pairs of first plasma emitters 21 are arranged on the side wall, deviating from the ventilation assembly 5, of the deflector 74, so that the plurality of pairs of first plasma emitters 21 are arranged above the filter assembly 3, the layout of the components in the purifiers is more reasonable, and the space in the purifier is utilized better.

The ventilation assembly 5 includes a wind wheel 51, a driving device 52, and a pressing cover 53. In the present embodiment, the wind wheel 51 is a centrifugal wind wheel, which facilitates the dispersion of air in the ventilation channel 65. The driving device 52 drives the wind wheel 51 to rotate, a plurality of air inlet channels 511 are formed in the wind wheel 51, and the air of the lower shell 67 enters the from the air inlet channels 511 and is dispersed to the air outlet chamber 64. The wind wheel 51 is arranged at a deflector 633, and the wind wheel 51 covers the air outlet 62, facing the air outlet chamber 64, of the ventilation channel 65. The pressing cover 53 is arranged to compress the driving device 52, so as to prevent the driving device 52 from shaking.

The flow guide frame 71 is arranged between the first mounting frame 72 and the air outlet 62, and the ventilation channel 65 is formed on the periphery of the flow guide frame 71. The flow guide frame 71 is in a circular truncated cone shape. In the air outlet direction X of the ventilation channel 65, the radial width of a lower bottom surface of the flow guide frame 71 is greater than the radial width of an upper top surface of the flow guide frame 71. The lower bottom surface of the flow guide frame 71 faces the ventilation assembly 5. The upper top surface of the flow guide frame 71 faces the air outlet 62. The ventilation assembly 5 pumps the air in the air inlet chamber 63 to the air outlet chamber 64. The upper top surface with a smaller radial width of the flow guide frame 71 faces the air outlet 62, while the lower bottom surface with a larger radial width of the flow guide frame 71 faces the ventilation assembly 5, so that in the air outlet direction X of the ventilation channel 65, the air pumped by the ventilation assembly 5 is gradually gathered at the periphery of the flow guide frame 71 along a side conical surface of the flow guide frame 71 and flows to the air outlet 62, and the air dispersed by the ventilation assembly 5 is gathered under the flow guiding effect of the flow guide frame 71, which increases the air outlet volume.

The first mounting frame 72 is arranged between the flow guide frame 71 and the ventilation assembly 5. A flow guide section 721 is formed at one end, facing the ventilation assembly 5, of the first mounting frame 72. The flow guide section 721 is shaped like a circular truncated cone. In the air outlet direction X of the ventilation channel 65, the radial width of the flow guide section 721 increases gradually. The lower bottom surface of the flow guide frame 71 is connected to the side wall, facing the flow guide frame 71, of the first mounting frame 72. The radial width of the lower bottom surface of the flow guide frame 71 is equal to the maximum radial width of the flow guide section 721.

In the present embodiment, the first mounting frame 72 is provided with a connecting section 722. The connecting section 722 is arranged between the flow guide frame 71 and the flow guide section 721. The connecting section 722 and the flow guide section 721 are integrally connected. The connecting section 722 is a circular cylinder. The radial width of the connecting section 722 is equal to each of the radial width of the lower bottom surface of the flow guide frame 71 and the maximum radial width of the flow guide section 721. The lower bottom surface of the flow guide frame 71 is adjacent to the connecting section 722. The driving device 52 and the pressing cover 53 are arranged in the flow guide section 721. The wind wheel is arranged outside the flow guide section 721. The driving device 52 drives the wind wheel 51 to rotate after penetrating through the flow guide section 721. The driving device 52 is an electric machine or a motor. A plurality of blades 512 are arranged on the wind wheel 51. An air inlet channel 511 is formed between two adjacent blades 512. The wind wheel 51 covers a ventilation opening 821, facing the air outlet chamber 64, of the air inlet chamber 63. The wind wheel 51 rotates to pump the air outside the purifier into the air inlet chamber 63, and then, the air is dispersed into the air outlet chamber 64 through the air inlet channel 511. In the present embodiment, one side, facing the flow guide section 721 of the first mounting frame 72, of the blade 512 is arc-shaped. The arc-shaped surface on the blade 512 corresponds to an inclined surface of the flow guide section 721, so that the arc-shaped surface on the blade 512 fits the inclined surface of the flow guide section 721 better, and the air flows better along the outer contour of the flow guide section 721, is dispersed to the periphery of the first mounting frame 72, and then climbs along the flow guide section 721 and the outer side wall of the flow guide frame 71.

The second mounting frame 73 is annular. The first mounting frame 72 is arranged in the inner circle of the second mounting frame 73. The second mounting frame 73 is adjacent to the inner side wall of the shell. The ventilation channel 65 is located between the first mounting frame 72 and the second mounting frame 73. The second mounting frame 73 is connected to the inner side wall of the middle shell 662.

Four bumps 7122 are formed in the outer side wall of the flow guide frame 71. The four bumps 7122 are arranged along the periphery of the flow guide frame 71 at equal distance. Four connecting blocks 75 are connected between the first mounting frame 72 and the second mounting frame 73. A mounting groove 751 is formed in each of connecting blocks 75, and one of the bumps 7122 is correspondingly arranged in one of the mounting grooves 751. The second mounting frame 73 is connected to the inner side wall of the shell 6, while the flow guide frame 71 is connected to the second mounting frame 73 through a structure that the bumps 7122 are in fit connection with the mounting grooves 751, so as to realize the mounting of the flow guide frame 71 in the shell 6. The plurality of connecting blocks 75 are connected between the first mounting frame 72 and the second mounting frame 73, which facilitates the penetrating of the ventilation channel 65 between the first mounting frame 72 and the second mounting frame, so that the air flows along the periphery of the flow guide shuttle structure formed by the flow guide section 721, the connecting section 722, and the flow guide frame 71.

In the present embodiment, the flow guide frame 71 includes an upper flow guide frame 711 and a lower flow guide frame 712. The upper flow guide frame 711 and the lower flow guide frame 712 are respectively circular truncated cones. In the vertical direction, the upper flow guide frame 711 is arranged below the lower flow guide frame 712. In the present embodiment, the radial width of the bottom surface, adjacent to the lower flow guide frame 712, of the upper flow guide frame 711 is equal to the radial width of the top surface, adjacent to the lower flow guide frame 712, of the upper flow guide frame 711. The bottom surface, facing the first mounting frame, of the lower flow guide frame 712 is the lower bottom surface of the flow guide frame 71. The radial width of the wind wheel is not greater than the radial width of the lower bottom surface of the flow guide frame 71. The upper flow guide frame 711 and the lower flow guide frame 712 can be connected through a plurality of buckle structures 713. The bumps 7122 are formed on the lower flow guide frame 72.

The second plasma generator 4 includes a plurality of pairs of second plasma emitters 41 and a high-voltage power assembly 42. The plurality of pairs of second plasma emitters 41 are arranged on the outer side wall of the upper flow guide frame 711 in the circumferential direction of the upper flow guide frame 711. Each pair of second plasma emitters 41 includes a positive ion emitter and a negative ion emitter. A first mounting chamber 7111 is formed in the upper flow guide frame 711. A high-voltage power supply assembly 42 of the second plasma generator 4 is arranged in the first mounting chamber 7111. A second mounting chamber 7211 is formed in the lower flow guide frame 712. A high-voltage power supply assembly 22 of the first plasma generator 2 and a control assembly 8 are arranged in the second mounting chamber 7211. The high-voltage power supply assembly 22 of the first plasma generator 2 is connected to the plurality of first plasma emitters 21. The control assembly 8 is connected to the first plasma generator 2 and the second plasma generator 4. Electrical elements or the control assembly 8 are respectively mounted in the first mounting chamber 7111 and the second mounting chamber 7211, which can reduce the influence of the mounting of the electrical elements on the ventilation volume of the ventilation channel 65, and meanwhile, realizes insulation protection on the electrical elements, so that the layout of the components in the shell 6 is more reasonable.

In the present embodiment, the shell 6 is in a circular truncated cone shape. In the vertical direction, from the bottom of the shell 6 to the top of the shell 6, the radial width of the shell 6 decreases gradually, so that the radial width of the air outlet chamber 64 decreases gradually in the air outlet direction X of the ventilation channel 65. In the vertical direction, the air outlet 62 is provided above the air inlet 61. The width of the bottom of the shell 6 is greater than the width of the top of the shell 6, so that the shell 6 has a good anti-dumping effect.

When the air of the air inlet chamber 63 is pumped into the air outlet chamber 64 through the ventilation assembly 5, the air in the air inlet chamber 63 enters the air outlet chamber 64 through an air inlet channel between two adjacent blades 512. Under the action of the wind wheel 51, the air is dispersed in the ventilation channel 65. Under the guiding action of the deflector 74 and the flow guide section 721, the air turns smoothly at the connecting section 722. The air starts to guide, along an outer wall of the circular truncated cone-shaped flow guide frame 71, the air here to climb along an outer conical surface of the flow guide frame 71. Since the radial width of the flow guide frame 71 decreases gradually in the air outlet direction X of the ventilation channel 65, so that the circulation space of the ventilation channel 65 increased gradually. Under the guiding action of the middle shell 662 with the radial width decreasing gradually, the air pumped by the ventilation assembly 5 flows to the air outlet 62 after gradually gathering at the periphery of the flow guide frame 71 along the side conical surface of the flow guide frame 71, and the air dispersed by the ventilation assembly 5 is gathered to increase the air outlet volume under the guiding effect of the flow guide frame 71. The flow guide section 721, the connecting section 722, and the flow guide frame 71 form a flow guide shuttle structure. The flow guide shuttle structure is arranged at the center of the air outlet chamber 64. The air flow in the air outlet chamber 64 flows in the air outlet direction X of the ventilation channel 65 on the periphery of the flow guide shuttle structure. In a flowing process of the air in the air outlet chamber 64, the flow guide shuttle structure is located at the center of the air flow of the air outlet chamber 64, so that the center of the air flow is blocked by the flow guide shuttle structure, and the air is dispersed on the periphery of the flow guide shuttle structure and flows along the periphery of the flow guide shuttle structure, thereby preventing the air flow from forming a vortex at the center of the air flow during flowing, reducing the loss of air volume, effectively improving the air speed, and reducing the generation of noise.

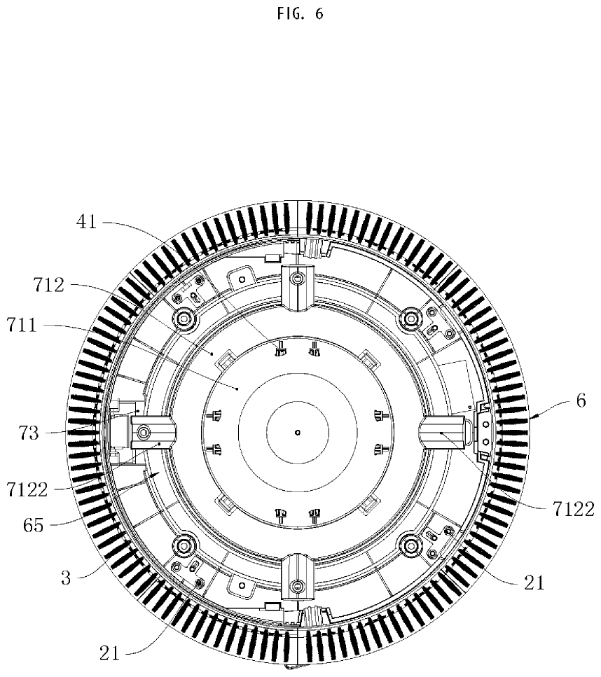

Referring to FIG. 6, in a first projection direction, the plurality of first plasma emitters 21 and the plurality of second plasma emitters 41 are arranged in the circumferential direction of the ventilation channel 65 in a staggered mode. By the structure, there is a second plasma emitter 41 between two adjacent first plasma emitters 21 in a pattern obtained after projection. In the circumferential direction of the air outlet chamber 64, an included angle between the first plasma emitter 21 and the adjacent second plasma emitter 41 is 45°. The first projection direction is the vertical direction. The plurality of pairs of emitters of the two plasma generators are arranged in a staggered mode, so that the plasmas finally released into a room are distributed more evenly, and the disinfection and sterilization effects are better.

An air outlet grille 621 is arranged at the air outlet 62. A plurality of air outlet holes 622 are formed in the air outlet grille 621. The side walls of the air outlet holes 622 are planes. The side walls of the air outlet holes 622 extend in the vertical direction. The air is guided by an inclined plane of a side conical surface of the flow guide frame 71 first in the air outlet chamber 65, and then passes through straight planes of the side walls of the air outlet holes 622. The side walls of the air outlet holes 622 guide the air flow, so as to reduce the cases that air encounters a turn during high-speed flow, thereby avoiding the resistance and noise generated due to turning, and realizing even dispersion.

A top plate 9 is arranged outside the air outlet 62. The top plate 9 is circular. The top plate 9 is arranged in a horizontal direction. The top plate 9 is connected to the shell 6. A control panel 91 is arranged on the top plate 9. A control assembly 92 is arranged in the top plate. The control assembly 92 is connected to each of the control panel 91 and the control assembly 8. The top plate 9 blocks outside the air outlet 62, so that the air exhausted from the air outlet 62 is released into the air through an outer edge of the top plate 9. Therefore, the air in the purifier is diffused to the periphery, and the plasma jet in indoor air is distributed more evenly.

Two plasma generators are arranged in the air purifier. The first plasma generator 2 is arranged close to the air inlet 61, and the plasma generator close to the air inlet 61 is arranged between the air inlet 61 and the filter assembly 3, so that a plasma wall is formed on the periphery of the filter assembly 3. In the air outlet direction of the ventilation channel 65, under the action of the ventilation assembly 5, air passes through the plasma wall formed by the first plasma generator 2 and the filter assembly 3 in sequence from the air inlet 61 along the horizontal air inlet section 651.

The air entering the purifier flows into the air outlet chamber 64 along the vertical air inlet section 652 after being disinfected and sterilized, while the plasmas released by the second plasma generator 4 form a plasma jet with the disinfected and sterilized air, and is dispersed into a room. With the work of the air purifier, positive ions and negative ions are continuously released into the air, and the positive ions and the negative ions in the indoor air outside the purifier produce and release huge energy at the moment of neutralizing positive and negative charges in the air, resulting in the change of a bacterial structure around or the conversion of energy, resulting in the death of bacteria. The plasma jet in the indoor air outside the purifier realizes a sterilization effect thereof. In the air circulation realized by the purifier, the two plasma generators respectively sterilize the air inside and outside the purifier, so that the air is purified more thoroughly.

Finally, it should be emphasized that the above is merely preferred embodiments of the disclosure and is not intended to limit the disclosure. For those skilled in the art, the disclosure may have various variations and changes. Any modifications, equivalent replacements, improvements and the like made within the spirit and principle of the disclosure shall fall within the scope of protection of the disclosure.

What is claimed is:

1. An air purifier, comprising a shell, a first plasma generator, a filter assembly, a second plasma generator, and a ventilation assembly, wherein an air inlet and an air outlet are formed in the shell; a ventilation channel is formed between the air inlet and the air outlet of the shell; the first plasma generator, the filter assembly, the second plasma generator, and the ventilation assembly are separately arranged in the ventilation channel; in an air outlet direction of the ventilation channel, the first plasma generator is arranged between the air inlet and the filter assembly, and the second plasma generator is arranged between the filter assembly and the air outlet.

2. The air purifier according to claim 1, wherein
the first plasma generator comprises a plurality of pairs of first plasma emitters, and the second plasma generator comprises a plurality of pairs of second plasma emitters; and in a first projection direction, the plurality of pairs of first plasma emitters and the plurality of pairs of second plasma emitters are arranged in a circumferential direction of the ventilation channel in a staggered mode.

3. The air purifier according to claim 1, wherein
in a vertical direction, the air outlet is provided above the air inlet; and/or
the shell comprises an air inlet chamber and an air outlet chamber; the air inlet chamber is arranged above the air inlet chamber; the air inlet chamber is communicated with the air inlet; the air outlet chamber is communicated with the air outlet; the ventilation assembly is arranged to send the air of the air inlet chamber into the air outlet chamber; the first plasma generator and the filter assembly are separately arranged in the air inlet chamber; and the second plasma generator is arranged in the air outlet chamber.

4. The air purifier according to claim 3, wherein
the shell comprises side plate and a base; the side plate is connected to the base along a circumferential direction of the base; a plurality of through holes are formed in the side plate; the plurality of through holes form the air inlet; a horizontal air inlet section is formed between the side plate and the filter assembly in the air inlet chamber; and
in the horizontal air inlet section, in an air outlet direction of the horizontal air inlet section, the first plasma generator is arranged between the side plate and the filter assembly.

5. The air purifier according to claim 4, wherein
in the vertical direction, the first plasma generator is arranged above the filter assembly; and
based on the base, the maximum arrangement height of the plurality of through holes in the side plate is greater than an arrangement height of the first plasma generator.

6. The air purifier according to claim 5, wherein
the filter assembly is in a shape of a circular cylinder, and a vertical ventilation section is formed in an inner circle of the filter assembly; in the air outlet direction of the ventilation channel, the vertical ventilation section is located downstream of the horizontal air inlet section;
the filter assembly comprises a first filter layer, a second filter layer, and a third filter layer; in a radial direction of the filter assembly, the second filter layer is wrapped outside the third filter layer, and the first filter layer is wrapped outside the second filter layer; and
the first filter layer is a nylon filter screen, the second filter layer is a High-Efficiency Particulate Air (HEPA) filter, and the third filter layer is an activated carbon filter layer.

7. The air purifier according to claim 1, wherein
the ventilation assembly covers a ventilation opening, facing the ventilation assembly, of the air inlet chamber.

8. The air purifier according to claim 1, wherein
a flow guide assembly is arranged in an air outlet chamber of the shell; and the flow guide assembly is arranged between the ventilation assembly and the air outlet.

9. The air purifier according to claim 8, wherein the flow guide assembly comprises a flow guide frame; the ventilation channel is formed on a periphery of the flow guide frame; the flow guide frame is in a circular truncated cone shape; in an air outlet direction of the ventilation channel, a radial width of a lower bottom surface of the flow guide frame is greater than a radial width of an upper top surface of the flow guide frame; the lower bottom surface of the flow guide frame faces the ventilation assembly; and the upper top surface of the flow guide frame faces the air outlet.

10. The air purifier according to claim 9, wherein
the flow guide assembly further comprises a first mounting frame; the first mounting frame is arranged between the flow guide frame and the ventilation assembly; a flow guide section is formed at one end, facing the ventilation assembly, of the first mounting frame; the flow guide section is shaped like a circular truncated cone; and in an air outlet direction of the ventilation channel, a radial width of the flow guide section increases gradually.

11. The air purifier according to claim 10, wherein
the lower bottom surface of the flow guide frame is adjacent to a side wall, facing the flow guide frame, of the first mounting frame; and the radial width of the lower bottom surface of the flow guide frame is equal to the maximum radial width of the flow guide section.

12. The air purifier according to claim 8, wherein
the flow guide assembly comprises a deflector; the deflector is annular; a first end, along the air outlet direction of the ventilation channel, of the deflector is arranged close to the air inlet chamber of the shell; a second end, along the air outlet direction of the ventilation channel, of the deflector is arranged away from the air inlet chamber; the first end of the deflector is arranged along a circumferential direction of the air inlet chamber close to the ventilation opening of the air outlet chamber; the second end of the deflector is arranged along a circumferential direction of the inner side wall of the shell; and
in the air outlet direction of the ventilation channel, the radial width of the deflector increases gradually from the first end of the deflector to the second end of the deflector.

13. The air purifier according to claim 12, wherein
the ventilation assembly is arranged in an inner circle of the deflector; and/or
the first plasma generator is arranged on the side wall, deviating from the ventilation assembly, of the deflector.

14. The air purifier according to claim 10, wherein
the flow guide assembly further comprises a second mounting frame; the second mounting frame is annular; the first mounting frame is arranged in an inner circle of the second mounting frame; the second mounting frame is adjacent to an inner side wall of the shell; and the ventilation channel is located between the first mounting frame and the second mounting frame.

15. The air purifier according to claim 14, wherein
at least two bumps are formed on an outer side wall of the flow guide frame;
at least two connecting blocks are connected between the first mounting frame and the second mounting frame; a mounting groove is formed in each of the at least two connecting blocks; and one of the bumps is correspondingly arranged in one of mounting grooves.

16. The air purifier according to claim 10, wherein
the ventilation assembly comprises a driving device and a wind wheel; the driving device drives the wind wheel to rotate; and the driving device is arranged in the inner circle of the flow guide section.

17. The air purifier according to claim 16, wherein
the wind wheel comprises a plurality of blades; an air inlet channel is formed between two adjacent blades of the plurality of blades; the ventilation channel penetrates through the air inlet channel; and the side wall, facing the flow guide section, of the blade is in an arc.

18. The air purifier according to claim 9, wherein the second plasma generator comprises a plurality of pairs of second plasma emitters, and the plurality of pairs of second plasma emitters are arranged on the flow guide frame;
the flow guide frame comprises an upper flow guide frame and a lower flow guide frame; the upper flow guide frame and the lower flow guide frame are respectively shaped like circular truncated cones; a lower bottom surface of the upper flow guide frame is relatively butted with an upper top surface of the lower flow guide frame; a radial width of the lower bottom surface of the upper flow guide frame is equal to a radial width of the upper top surface of the lower flow guide frame;
a first mounting chamber is formed in the upper flow guide frame; a high-voltage power supply assembly of the second plasma generator is arranged in the first mounting chamber;
a second mounting chamber is formed in the lower flow guide frame; a control assembly and a high-voltage power supply assembly of the first plasma generator is arranged in the second mounting chamber; and the control assembly is connected to the first plasma generator and the second plasma generator.

19. The air purifier according to claim 1, further comprising a flow guide assembly, wherein the shell, the ventilation assembly, and the flow guide assembly form an air channel assembly;
the shell further comprises an air inlet chamber and an air outlet chamber; the air inlet chamber is communicated with the air inlet; the air outlet chamber is communicated with the air outlet; the air inlet chamber is communicated with the air outlet chamber; the ventilation channel penetrates through the air inlet chamber and the air outlet chamber;
the ventilation assembly is arranged to send the air in the air inlet chamber into the air outlet chamber;
the flow guide assembly is arranged in the air outlet chamber; and the flow guide assembly is arranged between the ventilation assembly and the air outlet.

20. The air purifier according to claim 1, wherein
the air outlet chamber is in a circular truncated cone shape; and in the air outlet direction of the ventilation channel, a radial width of the air outlet chamber decreases gradually; and/or
the air outlet is located at a top of the shell, and in the vertical direction, the air outlet is provided above the air inlet; and/or
an air outlet grille is arranged at the air outlet; a plurality of air outlets are formed in the air outlet grille; a side walls of the air outlets are planes; and the side walls of the air outlets extend in a vertical direction; and/or
the shell is in a circular truncated cone shape; and a width of a bottom of the shell is greater than a width of a top of the shell.
